(19) United States
(12) Patent Application Publication
(10) Pub. No.: US 2012/0135441 A1
(43) Pub. Date: May 31, 2012

(54) ENZYMES AND METHODS FOR RESOLVING AMINO VINYL CYCLOPROPANE CARBOXYLIC ACID DERIVATIVES

(75) Inventors: Ian N. Taylor, Ely (GB); Michael C. Lloyd, Ely (GB); Adrian Heseltine, Leeds (GB)

(73) Assignees: DR. REDDY'S LABORATORIES, INC., Bridgewater, NJ (US); DR. REDDY'S LABORATORIES LTD., Hyderabad (IN)

(21) Appl. No.: 13/342,155

(22) Filed: Jan. 2, 2012

Related U.S. Application Data
(63) Continuation of application No. PCT/US2010/040900, filed on Jul. 2, 2010.
(60) Provisional application No. 61/222,767, filed on Jul. 2, 2009.
(60) Provisional application No. 61/222,772, filed on Jul. 2, 2009.
(60) Provisional application No. 61/223,023, filed on Jul. 4, 2009.

(51) Int. Cl. C12Q 1/37; C07C 227/18; C07C 229/48; C07C 227/16; C12Q 1/44; C12P 41/00

(52) U.S. Cl. 435/23; 435/19; 435/280; 560/124

(57) ABSTRACT

Preparation and isolation of amino vinyl cyclopropane carboxylic acid derivatives and salts thereof, methods of resolving enantiomers, and methods of identifying compositions and/or enzymes that are capable of resolving racemic or partially enantiomerically enriched mixtures.

### SEQUENCE LISTING

This application includes a sequence listing submitted herewith electronically as an ASCII text file created on Jul. 1, 2010, named “CPS3_{—}4-009_SequenceListing_ST25.txt,” which is 10,952 bytes in size, and it is hereby incorporated by reference in its entirety.

### INTRODUCTION

Aspects of the present invention relate to the preparation and isolation of amino vinyl cyclopropane carboxylic acid derivatives and salts thereof, methods of resolving enantiomers, and methods of identifying compositions and/or enzymes that are capable of resolving racemic or partially enantiomerically enriched mixtures. In aspects, the salts of amino vinyl cyclopropane carboxylic acid derivatives are utilized in a hydrolase-catalysed bioresolution process, without the need for additional buffering capacity, to produce enantiomerically enriched 1-amino-2-vinylcyclopropane carboxylic acid derivatives.

Chemical synthesis of many compounds fails to selectively produce a desired enantiomer, thus resulting in racemic or enantiomeric mixtures that must be separated or resolved before further processing. Amino vinyl cyclopropane carboxylic acid derivatives have been taught to be key intermediates for the preparation of inhibitors of the Hepatitis C virus NS3 protease. See P. L. Beaulieu et al., “Synthesis of (1R,2S)-1-Amino-2-vinylcyclopropanecarboxylic Acid Vinyl-ACCA) Derivatives: Key Intermediates for the Preparation of Inhibitors of the Hepatitis C Virus NS3 Protease,” Journal of Organic Chemistry, Vol. 70(15), pp. 5869-5879, 2005.

The Beaulieu et al. article teaches an approach to manufacture such derivatives, involving condensation of benzaldehyde with ethyl glycinate hydrochloride, followed by reaction with trans-1,4-dibromobut-2-ene, to form a racemic amino vinyl cyclopropane carboxylic acid ethyl ester (1). The amine functionality on this compound is then protected by addition of a BOC group (i.e., a —C(O)OC(CH₃)₃ group) on the nitrogen atom. The protected compound (2) is subjected to enzymatic resolution and optionally is converted to the tosylate salt. Beaulieu et al. teach that, when handling solutions of amino ester (1), solvent must be removed under reduced pressure at room temperature.

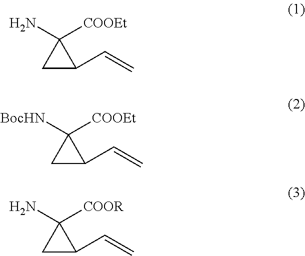

A direct resolution method, using unprotected compounds, will provide a simpler and more efficient route to such derivatives.

### SUMMARY

An alternative approach has been found for producing enantiomerically enriched amino vinyl cyclopropane carboxylic acid derivatives. Specifically, an approach involves a hydrolase catalysed bioresolution of amino ester (3).

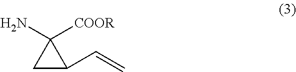

To obtain large quantities of an amino ester (3), one might consider distillation to separate the compound from solvent and reaction mixture, prior to enzymatic resolution. However, the room temperature vacuum separation of an amino ester (3) from solvents is not suitable for efficient large scale production. Moreover, it now has been discovered that 1-amino-2-vinylcyclopropane carboxylic acid esters, e.g., (3) where R is methyl or ethyl, have relatively low thermal stability, showing an exotherm at 50° C. under accelerated rate calorimetry (ARC). These factors limit processing options that would normally be desirable for use in large scale production.

The present applicants have discovered a number of novel salts that enable efficient large-scale production. Surprisingly, these salts have been found to have certain advantages, which allow a viable manufacturing process for enantio-enriched amino ester (3). These advantages include one or more of the following: enhanced thermal stability to temperatures beyond the melting point of the salt (typically >100° C.); avoidance of expensive BOC anhydride reactants; improved form of compound (solid, as opposed to the oil form of the free amine or the BOC protected amine) for better ease of handling and storage; avoidance of a time consuming low temperature vacuum separation step; avoidance of a distillation step that raises the temperature of the low thermal stability compound; and suitability for direct use of the salts in subsequent bioresolution steps, without the need for additional buffer.

In an aspect, the invention comprises a compound of formula (4),

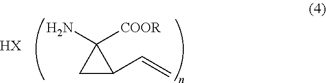

where R is an alkyl group, n is an integer of 1-3, and HX is an acid such as phosphoric acid, sulfuric acid, β,β-dimethylglutaric acid, citric acid, boric acid, acetic acid, maleic acid, malic acid, succinic acid, 3-(N-morpholino)propane sulfonic acid, 2-(N-morpholino)ethane sulfonic acid, 4-(2-hydroxyethyl)piperazine-1-ethanesulfonic acid, etc.

In an aspect, the invention comprises methods of making the above compound (4) by reacting a compound of formula (3) with an acid HX, as defined above.

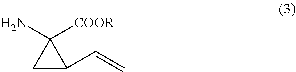

Another aspect of the invention comprises methods of making the above salt compound (4) by dialkylation of the appropriate (E)-N-phenylmethyleneglycine alkyl ester with trans-1,4-dihalobut-2-ene in a solvent, hydrolysis of the intermediate imino ester of formula (5) with an acid HX, and isolation of the resulting salt, such as by filtration.

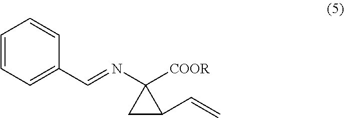

A further aspect of the invention comprises use of the above salt compound (4) in a hydrolase catalysed bioresolution process, without the need for additional buffer.

A further aspect of the present invention includes methods of identifying an enzyme capable of resolving a racemic or partially enantiomerically enriched mixture. Embodiments of a method include: providing a racemic or partially enantiomerically enriched mixture; exposing cell constituents to the racemic or partially enantiomerically enriched mixture; examining the racemic or partially enantiomerically enriched mixture for a change in the enantiomeric ratio; isolating an enzyme having resolving activity for the racemic or partially enantiomerically enriched mixture; and identifying said enzyme.

Aspects of the present invention include methods of resolving a racemic or partially enantiomerically enriched mixture of an ester of 1-amino-2-vinylcyclopropane carboxylic acid. Embodiments include: providing a racemic or partially enantiomerically enriched mixture of an ester of 1-amino-2-vinylcyclopropane carboxylic acid; and exposing said racemic or partially enantiomerically enriched mixture of an ester of 1-amino-2-vinylcyclopropane carboxylic acid to cell constituents.

### BRIEF DESCRIPTION OF THE DRAWINGS

FIG. 1 is a graphic representation of rates of hydrolysis of 50 g/L 1-amino-2-vinylcyclopropane carboxylic acid methyl ester using different enzymes, as measured using achiral high performance liquid chromatography (HPLC).

FIG. 2 is a sequence listing of a protein derived from Leuwenhoekiella blandensis and useful for producing enantiomerically enriched amino vinyl cyclopropane carboxylic acid derivatives.

FIG. 3 is a sequence listing of a protein derived from Crocibacter atlanticus and useful for producing enantiomerically enriched amino vinyl cyclopropane carboxylic acid derivatives.

### DETAILED DESCRIPTION

According to a first embodiment, this invention comprises a compound of formula (4),

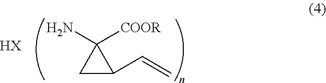

where R is an alkyl group. In embodiments, R has 1 to about 20 carbon atoms, or 1 to about 6 carbon atoms, or R is methyl or ethyl. In certain embodiments when R has more than 2 carbon atoms, R is an n-alkyl group. HX is an acid such as phosphoric acid, sulfuric acid, 3,3-dimethylglutaric acid, citric acid, boric acid, acetic acid, maleic acid, malic acid, succinic acid, 3-(N-morpholino)propanesulfonic acid, 2-(N-morpholino)ethanesulfonic acid, 4-(2-hydroxyethyl)piperazine-1-ethanesulfonic acid, etc., and n is an integer of about 1-3.

Salts can be made by reaction of an acid of formula HX to a solution containing a compound of formula (3).

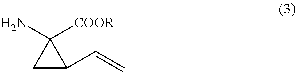

The concentration of the free amino ester (3) can be at least about 20 g/L, or at least about 50 g/L, or at least about 75 g/L, and generally less than about 200 g/L, or less than about 100 g/L. The amount of acid added is about 0.3-2 mole equivalents, or about 0.5-1.5 mole equivalents, or about 1 mole equivalent, per mole of amino ester.

The salt formation is undertaken in an organic solvent. A suitable solvent is one in which the free base (i.e., free amino ester) has good solubility, but in which the salt has low solubility. Useful solvents include, but are not limited to, ethers such as methyl t-butyl ether (MTBE), esters such as ethyl acetate and isopropyl acetate, halogenated hydrocarbons such as dichloromethane, and hydrocarbons such as toluene. Salt formation can be carried out using a combination of a solvent and a water soluble co-solvent (e.g., methanol, ethanol, acetone, and the like). The amount of the co-solvent is generally about 5-20%, based on the total volume of solvent.

Thus, according to embodiments, compound (4) may be made as illustrated in Scheme 1 below:

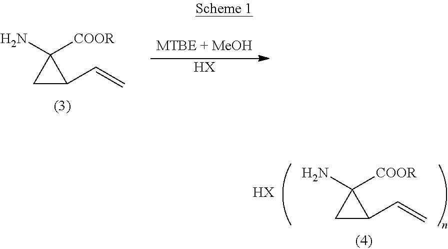

According to an approach, beginning with (E)-N-phenylmethylene glycine alkyl ester and trans-1,4-dihalobut-2-ene, the compound (4) may be made by dialkylation of the appropriate (E)-N-phenylmethyleneglycine alkyl ester with a trans-1,4-dihalobut-2-ene in solvent, hydrolysis of the intermediate imino ester using an acid, provided that if an acid other than a desired HX is used, the hydrolysis step is followed by adjusting the pH to about 8-9, solvent extraction, addition of a lower alcohol and acid HX; and isolation of the salt, such as by centrifugation, filtration, decantation, etc. This approach is illustrated in Scheme 2 below, which shows certain specific reagents.

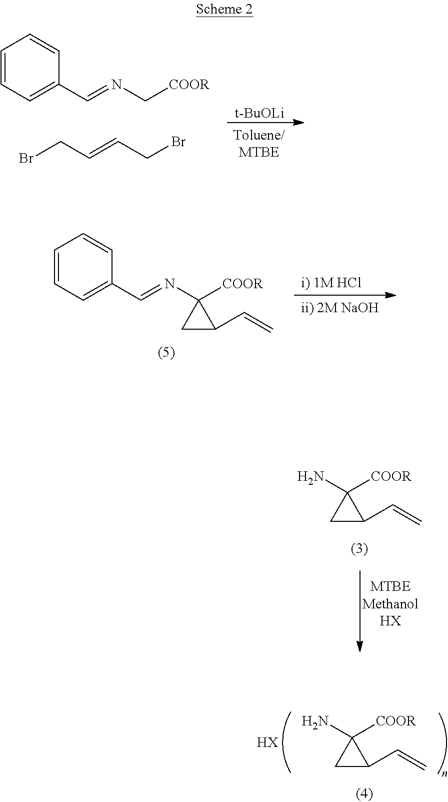

The dialkylation step is facilitated by bases. Useful bases include, but are not limited to, potassium hydroxide, sodium t-butoxide, potassium t-butoxide, lithium t-butoxide, lithium hexamethylsilazane, sodium hexamethylsilazane and potassium hexamethylsilazane, and the like. The trans-1,4-dihalobut-2-ene can be trans-1,4-dibromobut-2-ene.

The dialkylation step typically occurs in a suitable solvent. Non-limiting examples of such solvents are toluene, MTBE, hexane, and tetrahydrofuran (THF). An example of a useful solvent is a mixture of toluene and MTBE, containing 50-70% by volume MTBE.

Useful amounts of lithium t-butoxide or other base, per mole of trans-1,4-dibromobut-2-ene, are about 2.1-2.6 mole equivalents, Useful amounts of the (E)-N-phenylmethyleneglycine alkyl ester, per mole of trans-1,4-dibromobut-2-ene, are about 1.05-1.5 mole equivalents.

The solution of the imino ester (5) resulting from the dialkylation step is then hydrolyzed. According to one approach, the hydrolysis step comprises using an appropriate acid such as, but not limited to, hydrochloric acid, sulfuric acid, nitric acid, or phosphoric acid, including aqueous HCl in concentrations of 0.1M to 12M, or 2M to 6M.

After hydrolysis, the organic phase is discarded and base is added to raise the pH to about 8-9. Suitable bases include, but are not limited to, sodium hydroxide, potassium hydroxide, sodium carbonate, and sodium bicarbonate. The amino ester may then be extracted into a suitable organic solvent, such as MTBE. In this approach (using an acid other than a desired HX), one then adds an acid HX to form the salt according to the procedure set forth above.

According to an alternate approach illustrated in Scheme 3, the hydrolysis of 5 can be undertaken directly with HX to form the corresponding salt. The amount of acid HX added, per mole of amino ester, in this approach is in the range of about 0.3-2 mole equivalents, or about 0.5-1.5 mole equivalents, or about 1 mole equivalent.

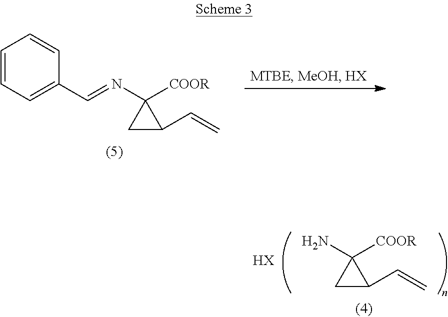

The salt is then isolated, such as by centrifugation, filtration, decantation, etc., as a solid that is thermally stable and can be easily handled for future reactions. The compound (4) can be used in enzymatic resolution of the racemic species to preferentially obtain a desired single enantiomer form, as represented in Scheme 4.

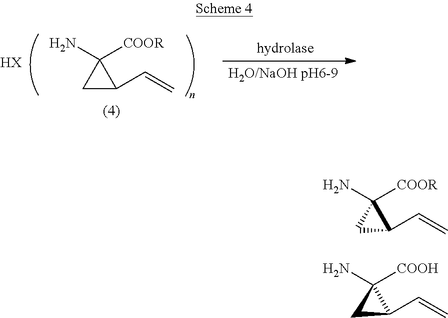

These salts can be used directly in a hydrolase catalysed enzymatic resolution by dissolution of the salt in water, adjustment of pH to the range of 6-9 by addition of base, and addition of a hydrolase enzyme. No additional buffer is needed. Examples of organisms from which suitable enzymes can be obtained include Formosa sp., Psychroserpens sp., Shewanella sp., Winogradskyella sp., Leeuwenhoekiella blandensis, Croceibacter atlanticus and Leeuwenhoekiella aequorea and Aquamarina, sp.

In a further aspect the present invention relates to methods of identifying compositions and/or enzymes capable of resolving racemic or partially enantiomerically enriched mixtures.

In embodiments, methods of identifying compositions and/or enzymes capable of resolving a racemic or partially enantiomerically enriched mixture of an ester of 1-amino-2-vinylcyclopropane carboxylic acid, as schematically represented in scheme 5, are provided.

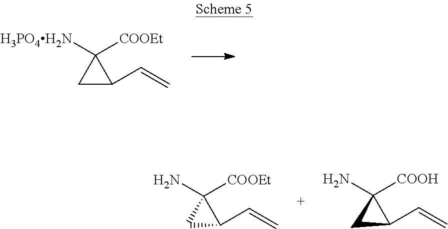

In particular embodiments, racemic or partially enantiomerically enriched mixtures can be exposed to cell constituents from one or more organisms. In further embodiments, racemic or partially enantiomerically enriched mixtures can be examined to determine if there are changes in the enantiomeric ratio or resolution of the mixtures.

In alternative embodiments, cell constituents shown to have resolving activity can be fractionated or separated and can be further tested for resolving activity, so as to isolate or identify one or more enzymes having the resolving activity. In additional embodiments, one or more enzymes having resolving activity can be, by way of non-limiting examples, in addition to an enzyme, a peptide or an RNA. In certain embodiments of the invention, a gene encoding one or more enzymes having resolving activity may be identified and cloned using techniques standard in the art. See, e.g., J. Sambrook et al. (eds), Molecular Cloning: A Laboratory Manual, Cold Spring Harbor Laboratory Press, 1.84-1.88, 2001.

Resolution, as used herein, relates to a change in the level of one of a pair of enantiomers relative to the other (the enantiomeric ratio). Thus, resolution might result from the modification of one enantiomer, thus making it no longer part of an enantiomeric pair, or the conversion of one enantiomer into the other enantiomer. One non-limiting example of resolution includes the cleaving of an ester to form 1-amino-2-vinylcyclopropane carboxylic acid.

Enantiomerically enriched, as used herein, refers to mixtures comprising a pair of enantiomers wherein the enantiomeric ratio is other than 1:1.

In embodiments of the invention, a racemic or enantiomerically enriched mixture may include any composition or solution containing the two species of an enantiomeric pair. In further embodiments, one or more groups of the enantiomeric pair may be protected by, for example, a BOC group. In additional embodiments, the molecules of the enantiomeric pair can be, or can exist as, part of a salt, such as, by way of non-limiting examples, a phosphate, sodium, nitrate, or calcium salt. In further embodiments, the molecules of the enantiomeric pair can be in the free amine form. In particular embodiments, the mixture or solution comprising the enantiomeric pair may comprise any solvent or solution. Examples of solvents or solutions include, but are not limited to, water, saline, buffered saline, phosphate buffered saline, and/or solutions comprising a polysorbate surfactant (e.g., a TWEEN® product).

In particular embodiments of the invention, exposing a racemic or partially enantiomerically enriched mixture to cell constituents may include any method or technique for bringing the mixture and the cell constituents in contact with each other, such that the cell constituents may at least partially resolve the mixture. Examples of methods of exposure include, but are not limited to, fluid contact and physical contact.

Exposure to cell constituents may take place for any period of time required to recognize or determine a statistically significant change in the enantiomeric ratio. Examples of time periods of exposure include, but are not limited to, from about 0.1 hours to about 72 hours, about 1 hour to about 48 hours, about 8 hours to about 30 hours, about 30 hours, and about 12 hours.

Exposure may also take place at any temperatures. In embodiments, exposure occurs approximately at temperatures that are the normal Jiving environment of the organisms from which the cell constituents are obtained. Examples of temperatures at which exposure may occur include, but are not limited to, about 1° C. to about 99° C., about 10° C. to about 50° C., and about 30° C.

Exposure to cell constituents may take place at any pH values. Examples of pH values at which exposure may occur include, but are not limited to, about pH 1 to about pH 12, about pH 3 to about pH 11, about pH 6 to about pH 9, about pH 9, and about pH 7.

In certain embodiments of the invention, cell constituents can include, but are not limited to, cell extracts, cell pastes, cell lysates, cell free extracts, lyophilized cell free extracts, lyophilized cell extracts, lyophilized cell pastes, lyophilized cell lysates, sonicated cells, isolated proteins, and/or combinations, and/or fractions, and/or fragments thereof.

The cell constituents can be from any organism or a combination of organisms. Examples of organisms from which cell constituents might be obtained include, but are not limited to, animals, plants, bacteria, archea, fungi, marine organisms, marine algae, Formosa sp., Psychroserpens sp., Shewanella sp., Winogradskyella sp., Leeuwenhoekiella blandensis, Croceibacter atlanticus and Leeuwenhoekiella aequorea, Aquamarina, sp., AQP317, and AQP383.

AQP317 was deposited with the National Collection of Industrial, Food and Marine Bacteria, Aberdeen, Scotland (“NCIMB”), under the Budapest treaty, as NCIMB 41475 on Mar. 9, 2007, and AQP383 was deposited with NCIMB as NCIMB 41476 on Mar. 9, 2007.

Leewenhoekiella blandensis was deposited with NCIMB, under the Budapest Treaty, in 2010. Croceibacter atlanticus was deposited with NCIMB, under the Budapest Treaty, in 2010. Leewenhoekiella aquorea was deposited with the NCIMB, under the Budapest Treaty, in 2010.

In alternative embodiments of the invention, cell constituents may be further fractionated or separated. Methods of fractionation and separation include, but are not limited to, various forms of chromatography, size exclusion, gel electrophoresis, iso-electric and precipitate separations. Cell constituents can be fractionated by ammonium sulfate precipitation.

In additional embodiments, enzymes having resolving activity can preferentially precipitate at ammonium sulfate concentrations of approximately 30% to 40%, or higher, and/or can preferentially precipitate at ammonium sulfate concentrations of approximately 50% to 60%, or higher.

The following examples will further illustrate certain specific aspects and embodiments. These examples are provided only for purposes of illustration, and should not be construed as limiting the scope of the invention in any manner.

### EXAMPLE 1

### Synthesis of 1-amino-2-vinylcyclopropanecarboxylic Acid Methyl Ester

To a stirred solution of trans-1,4-dibromo-2-butene (340.6 g, 1.33 mol) in MTBE (1.5 L) is added lithium tert-butoxide (318 g, 3.325 mol). The resulting suspension is cooled below 15° C. and a solution of (E)-N-phenylmethyleneglycine methyl ester (310 g, 1.75 mol) in toluene (875 mL) is slowly added over 60 minutes, ensuring that the reaction temperature remains at 15-20° C. After stirring for an additional 2 hours at 20-25° C., the reaction is quenched by adding NaCl solution (20% by weight, 2 L). The organic phase is mixed with 1M HCl solution (1.75 L, 1.75 mol) and vigorously stirred at 20° C. for 2 hours. Aliquots are taken from the mass to ensure that all of the intermediate imine has been hydrolysed. Phases are then separated and the aqueous phase is washed with 500 ml MTBE. The aqueous phase is then cooled to 13° C. and pH is adjusted to about 9 with NaOH solution (6M, 200 mL). The mixture is then extracted with MTBE (5 L), yielding a solution containing 186 g of 1-amino-2-vinylcyclopropanecarboxylic acid methyl ester.

### EXAMPLE 2

### Synthesis of 1-amino-2-vinylcyclopropanecarboxylic Acid Methyl Ester Phosphate Salt

Into a round bottom flask equipped with an overhead stirrer is placed a solution of 1-amino-2-vinylcyclopropane carboxylic acid methyl ester (7 g) in MTBE (110 mL). Methanol (10 mL) is then added and the mixture is stirred at room temperature. Orthophosphoric acid (3.5 mL) is added drop-wise and a precipitate forms. After the addition is complete, stirring is continued for a further 60 minutes. The phosphate salt is then recovered by filtration and dried in vacuo. The phosphate salt is obtained as a beige powder in a yield of 11.72 g (˜98%).

¹H NMR (D₄-MeOH): δ 5.82-5.70 (m, 1H), 5.42 (dd, J=17 & 1, 1H), 5.22 (dd, J=10 & 1, 1 H), 3.86 (s, 3H), 2.49 (q, J=9, 1H), 1.85-1.80 (m, 1 H), 1.80-1.73 (m, 1H).

### EXAMPLE 3

### Synthesis of 1-amino-2-vinylcyclopropane Carboxylic Acid Methyl Ester Phosphate Salt

In a glass-lined reactor, a solution of approximately 1-amino-2-vinyl cyclopropane carboxylic acid methyl ester (70 Kg) in MTBE (566 Kg) is prepared via the synthesis procedure described in Example 1. After drying the solution with magnesium sulphate and filtering, methanol (40 Kg) is added. At a temperature below 15° C., 80% phosphoric acid (60 Kg) is added gradually, over 40 minutes. During this time a precipitate is formed. Once the addition is complete, the slurry is stirred below 10° C. for at least an additional hour. The solid is collected by filtration, washed with MTBE, and discharged from the filter to storage containers as an off-white damp filter cake with typically 23% MTBE content. About 138.5 kg of damp filter cake is isolated, equivalent to about 106.5 kg (-90% yield) of 1-amino-2-vinylcyclopropane carboxylic acid methyl ester phosphate, on a solvent-free basis.

### EXAMPLE 4

### Synthesis of 1-amino-2-vinylcyclopropane Carboxylic Acid Ethyl Ester Phosphate Salt

Into a round bottom flask is placed a solution of 1-amino-2-vinyl cyclopropane carboxylic acid ethyl ester (8.5 g) in MTBE (100 mL). Methanol (12 mL) is then added and the mixture is stirred at room temperature. Orthophosphoric acid (6 mL) is added drop-wise and a precipitate starts to form. After the addition is complete, stirring is continued for a further 2 hours. The phosphate salt is recovered by filtration, washed with MTBE, and dried in vacuo. About 9.4 g (70% yield) of an off-white solid is obtained.

¹H NMR (D₄-MeOH): δ 5.81-5.67 (m, 1H), 5.38 (dd, J=16 & 1, 1H), 5.19 (dd, J=11 & 1, 1 H), 4.28 (q, J=7, 2H), 2.43 (q, J=9, 1 H), 1.80-1.68 (m, 2H), 1.31 (t, J=7, 3H).

### EXAMPLE 5

### Synthesis of 1-amino-2-vinylcyclopropane Carboxylic Acid Methyl Ester Phosphate Salt From N-phenyl Methylene Glycine Methyl Ester

To lithium t-butoxide (31.8 g) slurried in MTBE (50 mL) is added trans-1,4-dibromo-2-butene (34 g) dissolved in MTBE (100 mL). To the stirred reaction mixture is then added a solution of N-phenyl methyleneglycine methyl ester (31 g) in toluene (90 g). The temperature is maintained below 15° C. during the addition and subsequently the reaction is stirred at ambient temperature for 2 hours. The reaction is quenched by adding NaCl solution (20% by weight, 200 mL). The aqueous phase is discarded and to the organic phase is added approx 15% by volume of methanol. The solution is cooled below 5° C. and a molar equivalent of 85% phosphoric acid is added slowly to precipitate the phosphate salt. The mixture is stirred for 60 minutes, and precipitate is recovered by filtration and washed with MTBE. An amount of 32.3 g of 1-amino-2-vinylcyclopropane carboxylic acid methyl ester phosphate salt (85% yield, based on trans-1,4-dibromo-2-butene) with purity about 97% by HPLC is obtained.

### EXAMPLE 6

### Esterase Catalysed Bioresolution of 1-amino-2-vinylcyclopropane Carboxylic Acid Ethyl Ester Phosphate Salt

Into a 20 mL stem block tube is placed 1-amino-2-vinylcyclopropane carboxylic acid ethyl ester phosphate salt (0.5 g, 2 mmol) dissolved in deionised water (4 mL). The pH of the solution is adjusted to 8 by drop-wise addition of 1M NaOH solution (2 mL). The mixture is continuously stirred at 25° C. and lyophilised AQP317 (Formosa algae) (200 mg) is added. Stirring is continued at 25° C. for 41 hours, after which HPLC analysis determines that conversion has reached about 50% and gas chromatography (GC) analysis indicates that the enantiomeric excess of the residual ester has reached 99%.

### EXAMPLE 7

### Primary Screen for Esterase Activity

Substrate, 1-amino-2-vinyl-cyclopropane carboxylic acid methyl ester, (200 μL, 10 g/L in phosphate buffered saline) is applied to screening plates containing 1 mg per well of lyophilized cell paste. After overnight incubation at 30° C., the reactions are sampled into HPLC mobile phase and analyzed for amino acid formation. Further analysis of suspected hits is performed by analysis of residual ester as a trifluoroacetate by chiral GC on a Chirasil Dex CB column, helium carrier gas at 830 KPa (20 p.s.i.), oven temperature isothermal at 100° C. From a screen of 230 marine microorganisms, twelve confirmed hits are identified, wherein the residual ester enantiomeric excess is greater than 90%.

### EXAMPLE 8

### Aquapharm™ Organisms Screen

Approximately 4 mg of a subset consisting of 8 of the 12 confirmed hits of lyophilized organism preparations (obtained from Aquapharm Biodiscovery Ltd.) is weighed into glass scintillation vials along with 1 mL of 10 g/L 1-amino-2-vinyl-cyclopropane carboxylic acid methyl ester, in phosphate buffered saline+0.1% by volume Tween 80 (pH 7), or in phosphate buffer pH 9. These mixtures are incubated for 30 hours at 30° C., and 300 rpm. Post-reaction residual ester is analyzed by GC as a trifluoroacetic acid derivative. The results are shown in Table 1. All samples are more active at pH 7 than pH 9.

AQP250 is found to not be viable when recovery is attempted from primary culture plates. An alternative organism, identified as a Psyhcroserpens sp, and designated AQP 383, having similar morphology and isolated from the same original source, is used as an alternative. When assayed, this is also demonstrated to have the desired activity.

TABLE 1

| | Residual Ester Enantiomeric Excess | | | | | | |
|---|---|---|---|---|---|---|---|
| | pH 7 | | | | pH 9 | | |
| | 3 | 23 | 46 | 96 | 3 | 23 | |
| Organism | Hours | Hours | Hours | Hours | Hours | Hours | |
| AQP029 | | 15% | ND | ND | | 2% | Shewanella baltica |
| AQP246 | 0% | 11% | ND | ND | −3% | 1% | Shewanella baltica |
| AQP237 | 0% | 9% | ND | ND | −2% | 0% | |
| AQP250 | 2% | 32% | ND | ND | −1% | 2% | |
| AQP272 | 0% | 7% | 14.6% | 55.8% | −1% | 5% | Winogradskyella |
| | | | | | | | thalassocola |
| AQP317 | 2% | 25% | 60.4% | 93.5% | 1% | 22% | Formosa algae |
| AQP331 | 2% | 17% | 39.1% | 92.0% | −10% | 10% | Winogradskyella |
| | | | | | | | thalassocola |
| AQP332 | 1% | ND | ND | ND | −2% | ND | Aquamarina sp. |

### EXAMPLE 9

### Activity at 50 g/L Substrate Concentration

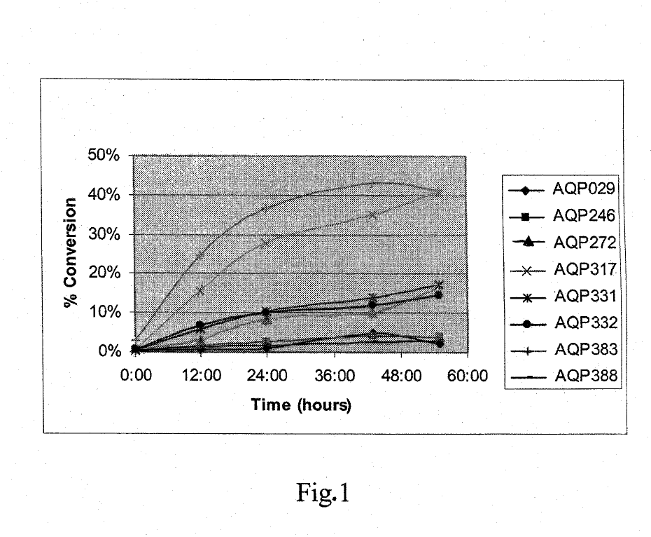

Reactions containing 200 mg of lyophilized cell paste and 250 mg of methyl-1-amino-2-vinyl-cyclopropane carboxylate-phosphate salt are prepared in a total of 5 mL of phosphate buffered saline and monitored for conversion over time using achiral HPLC. Two organisms, AQP317 and AQP383, demonstrate significantly greater rate of activity compared to the other confirmed hits, as judged by achiral HPLC, and completely resolve the substrate at these concentrations. The results are plotted in FIG. 1.

### EXAMPLE 10

### Alcalase-catalyzed Bioresolution of 1-amino-2-vinylcyclopropane Carboxylic Acid Ethyl Ester Phosphate Salt

1-Amino-2-vinylcyclopropane carboxylic acid ethyl ester phosphate salt (2 g, 7.9 mmol) in deionized water (45 mL) is placed into a jacketed vessel and pH is adjusted to 8 by adding 2M sodium hydroxide solution (5 mL). The mixture is continuously stirred at 35° C. and Alcalase® enzyme solution sold by Novozymes (6 mL) is added. The mixture is continuously stirred and pH is maintained at 8.15. Aliquots (100 μL) are periodically taken and GC analysis indicates e.e.=5.7% (t=24 hours), e.e.=14.3% (t=48 hours), and e.e.=21.4% (t=72 hours). No significant conversion has occurred after 72 hours and the reaction is halted. The reaction is represented in Scheme 5.

### EXAMPLE 11

### Preparation of Cell-free Extract of Formosa algae AQP317

A cell free extract of AQP317, Formosa algae, is prepared by re-suspending 500 mg of lyophilized cell paste, ex of 500 mL culture, in 50 mL of PBS. The cell suspension is sonicated, 15 μm amplitude, for 30 minutes with 10 seconds on and 15 seconds off, at 4° C. Debris is removed by centrifugation at 10,000 G for 10 minutes at 4° C.

The cell-free extract is subjected to a series of ammonium sulfate precipitations, precipitate is recovered by centrifugation at 10,000 G for 20 minutes at 4° C. See R. M. C. Dawson et al., eds., Data for Biochemical Research, Third Edition, pp 537-539, 1986. Each pellet is re-suspended in 5 mL of phosphate buffered saline (Sigma P4417). Protein content is assayed using Coomassie Plus reagent from Pierce. A similar experiment is performed using Psychroserpens AQP383, in which activity is demonstrated to precipitate at 30-40%.

### EXAMPLE 12

### Biotransformation Assay

Activity is assayed in 1 mL scintillation vials containing 10 mM 1-amino 2-vinylcyclopropane carboxylic acid ethyl ester phosphate salt in phosphate buffered saline. Reactions are sampled after 2.5 hours, and diluted 1:5 into a HPLC mobile phase prior to analysis. Percentage conversion is then used to calculate the number of enzyme activity units present, with results as shown in Table 2.

### EXAMPLE 13

### Isolation of Resolving Activity

Cell free extracts of AQP317 and AQP383 are fractionated using standard techniques. Various fractions are exposed to a racemic or partially enantiomerically enriched mixture of an ester of 1-amino-2-vinylcyclopropane carboxylic acid and incubated at 30° C. for 30 hours. The different fractions are then tested for alteration of the enantiomeric ratio, as previously described. Fractions having resolving activity are further fractionated and/or separated via gel electrophoresis. Further fractions and gel isolates are tested for resolving activity. A single compound having resolving activity is isolated and identified as an esterase. The isolated esterase is subjected to carboxyl and/or amino peptide sequencing or protein mass spectrometry. The sequence information is then used to generate putative primer pairs for the isolation of the gene encoding the esterase or to enable synthesis of the whole gene. DNA extracts of AQP317 and AQP383, Leuwenhoekiella blandensis, Croceibacter atlanticus, and Leuwenhoekiella aquorea are created and PCR is performed using the putative primer pairs. The PCR products are analyzed and the sequence encoding the esterase is isolated and cloned into a vector before sequencing.

### EXAMPLE 14

### Demonstration of Activity of the Cloned Polypeptide

A nucleotide sequence encoding the polypeptide is cloned into a Pseudomonas expression system, using standard techniques known to those skilled in the art.

Cultures of the recombinant esterase are grown at 25° C., induced after 24 hours growth and 1 mL samples are taken at intervals post induction and micro centrifuged. Pellets are resuspended in 1 mL of 20 g/L 1-amino-2-vinylcyclopropane carboxylic acid methyl ester phosphate salt pH 7 and incubated at 25° C. After 144 hours reaction time, the residual ester is extracted with MTBE, derivitised with trifluoroacetic anhydride, and analysed by gas chromatography. The residual ester has an enantiomeric excess of 87%.

TABLE 2

| | Whole | Whole | Sonicate | | | | | | | |
|---|---|---|---|---|---|---|---|---|---|---|
| Fraction | Cells | Sonicate | S/N | 0-20 | 20-30 | 30-40 | 40-50 | 50-60 | 60-70 | 70+ S/N |
| Volume (mL) | 50 | 50 | 50 | 5 | 5 | 5 | 5 | 5 | 5 | 50 |
| Protein (mg/mL) | 10 | 0.71 | 0.71 | 0.17 | 0.13 | 0.11 | 0.15 | 3.51 | 1.44 | 0.09 |
| Total Protein (mg) | | | 35.5 | 0.8 | 0.6 | 0.5 | 0.7 | 17.6 | 7.2 | 4.6 |
| Protein Yield (%) | | | | 2 | 2 | 2 | 2 | 49 | 20 | 13 |
| Cumulative Protein | | | | 2 | 4 | 6 | 8 | 57 | 77 | 90 |
| Yield (%) | | | | | | | | | | |
| Conversion* | 17 | 15 | 21 | 1 | 1 | 1 | 1 | 10 | 6 | 5 |
| Volume Assayed (mL) | 0.5 | 0.5 | 0.5 | 0.05 | 0.05 | 0.05 | 0.05 | 0.05 | 0.05 | 0.5 |
| Volumetric Activity** | 0.0229 | 0.0194 | 0.0277 | 0.0165 | 0.0123 | 0.0089 | 0.0101 | 0.1288 | 0.0809 | 0.0070 |
| Total Units | 1.15 | 0.97 | 1.39 | 0.08 | 0.06 | 0.04 | 0.05 | 0.64 | 0.40 | 0.35 |
| Activity Yield (%) | | | | 6 | 4 | 3 | 4 | 46 | 29 | 25 |
| Cumulative Activity | | | | 6 | 10 | 14 | 17 | 64 | 93 | 118 |
| Yield (%) | | | | | | | | | | |

*Percent at 2.5 hours.
**Units are μmol/minute/mL.

1. A compound of formula (4), 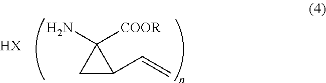
where R is an alkyl group, n is an integer of 1-3, and HX is phosphoric acid, sulfuric acid, β,β-dimethylglutaric acid, citric acid, boric acid, acetic acid, maleic acid, malic acid, succinic acid, 3-(N-morpholino)propane sulfonic acid, 2-(N-morpholino)ethane sulfonic acid, or 4-(2-hydroxyethyl)piperazine-1-ethane sulfonic acid.

2. The compound of claim 1, wherein R is methyl or ethyl

3. The compound of claim 1, wherein R is methyl and HX is phosphoric acid

4. The compound of claim 1, wherein R is ethyl and HX is phosphoric acid

5. A method of making a compound of formula (4), comprising reacting a compound of formula (3) with an acid HX, wherein R is an alkyl group, n is an integer of 1-3, and HX is phosphoric acid, sulfuric acid, β,β-dimethylglutaric acid, citric acid, boric acid, acetic acid, maleic acid, malic acid, succinic acid, 3-(N-morpholino)propanesulfonic acid, 2-(N-morpholino)ethanesulfonic acid, or 4-(2-hydroxyethyl)piperazine-1-ethanesulfonic acid.

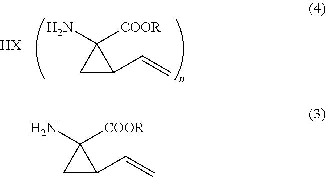

6. The method of claim 5, wherein reacting is conducted in an organic solvent.

7. The method of claim 5, wherein reacting is conducted in a solvent comprising MTBE, ethyl acetate, dichloromethane, isopropyl acetate, or toluene.

8. The method of claim 5, wherein reacting is conducted in a solvent comprising MTBE, ethyl acetate, dichloromethane, isopropyl acetate, or toluene, in combination with a water soluble co-solvent.

9. The method of claim 5, wherein the compound of formula (4) is isolated by filtration.

10. A method of making a compound of formula (4), comprising hydrolysing an imino ester intermediate of formula (5) with an acid HX, wherein R is an alkyl group, n is an integer of 1-3, and HX is phosphoric acid, sulfuric acid, β,β-dimethylglutaric acid, citric acid, boric acid, acetic acid, maleic acid, malic acid, succinic acid, 3-(N-morpholino)propanesulfonic acid, 2-(N-morpholino)ethanesulfonic acid, or 4-(2-hydroxyethyl)piperazine-1-ethanesulfonic acid.

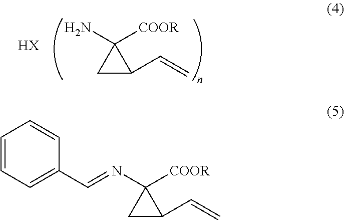

11. The method of claim 10, wherein the imino ester intermediate of formula (5) is obtained by dialkylation of (E)-N-phenylmethyleneglycine alkyl ester (6) with trans-1,4-dihalobut-2-ene of formula (7).

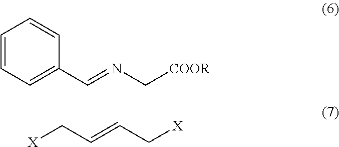

12. The method of claim 11, wherein the imino ester intermediate of formula (5) is hydrolysed without isolation, following the dialkylation.

13. The method of claim 10, wherein HX is phosphoric acid.

14. The process of claim 10, wherein the compound of formula (4) is isolated by filtration.

15. A method for hydrolase catalysed bioresolution, comprising using the compound of formula (4) in the bioresolution process without an additional buffer.

16. A method of identifying an enzyme capable of resolving a racemic or partially enantiomerically enriched mixture, comprising:
a) providing a racemic or partially enantiomerically enriched mixture;
b) exposing cell constituents to the racemic or partially enantiomerically enriched mixture;
c) examining the racemic or partially enantiomerically enriched mixture for a change in the enantiomeric ratio;
d) isolating an enzyme having resolving activity for the racemic or partially enantiomerically enriched mixture; and
e) identifying the enzyme.

17. The method of claim 16, wherein the racemic or partially enantiomerically enriched mixture is in a salt form.

18. The method of claim 16, wherein the racemic or partially enantiomerically enriched mixture is a phosphate, sodium, nitrate, or calcium salt.

19. The method of claim 16, wherein exposure is fluid contact.

20. The method of claim 16, wherein cell constituents are obtained from animals, plants, bacteria, archea, fungi, marine organisms, marine algae, Formosa sp., Psychroserpens sp., Shewanella sp., Winogradskyella sp., Leeuwenhoekiella blandensis, Croceibacter atlanticus and Leeuwenhoekiella aequorea, Aquamarina, sp., AQP317, and AQP383.

21. A method of resolving a racemic or partially enantiomerically enriched mixture of an ester of 1-amino-2-vinylcyclopropane carboxylic acid, comprising:
a) providing a racemic or partially enantiomerically enriched mixture of an ester of 1-amino-2-vinylcyclopropane carboxylic acid; and
b) exposing the racemic or partially enantiomerically enriched mixture of an ester of 1-amino-2-vinylcyclopropane carboxylic acid to cell constituents.

22. The method of claim 21, wherein cell constituents are obtained from animals, plants, bacteria, archea, fungi, marine organisms, marine algae, Formosa sp., Psychroserpens sp., Shewanella sp., Winogradskyella sp., Leeuwenhoekiella blandensis, Croceibacter atlanticus, Leeuwenhoekiella aequorea, Aquamarina sp., AQP317, and AQP383.

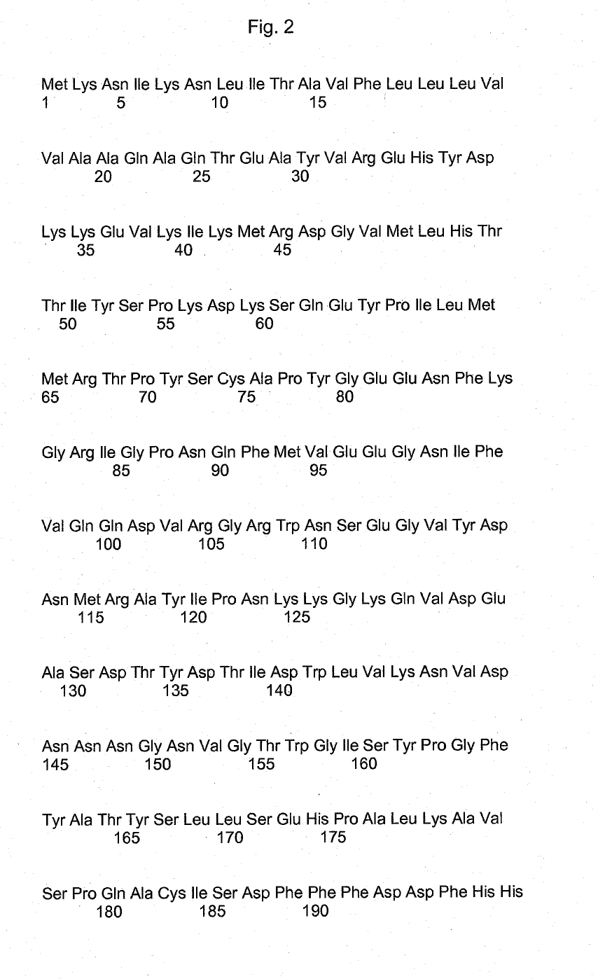

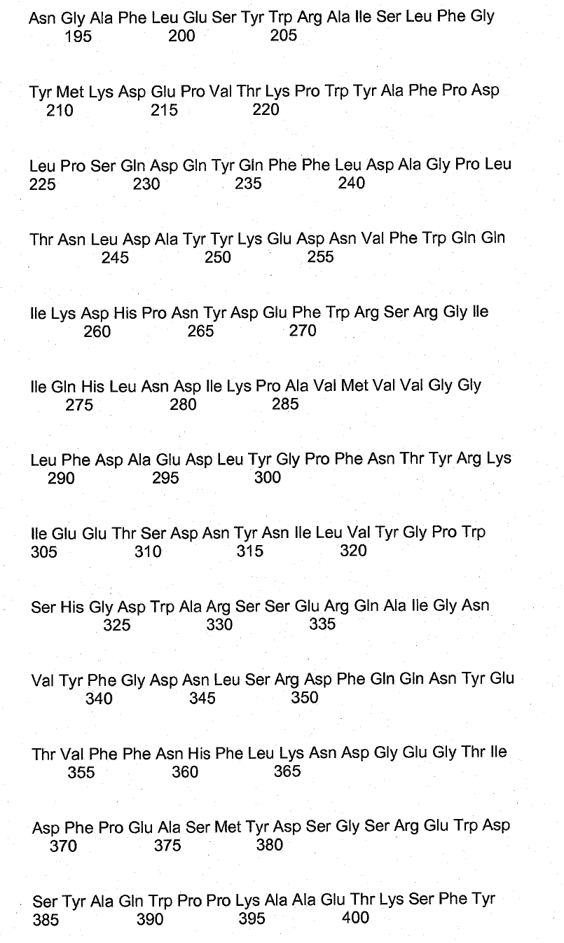

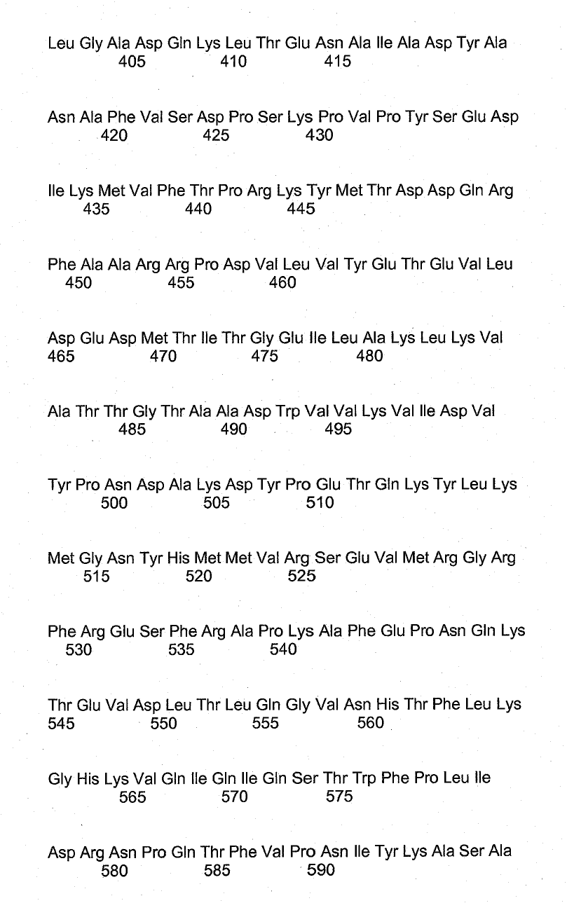

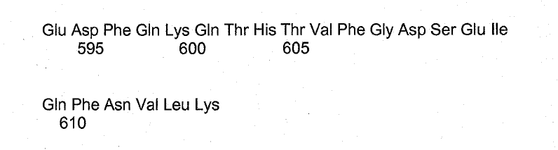

23. A method according to claim 20, wherein cell constituents comprise a protein having a sequence of FIG. 2.

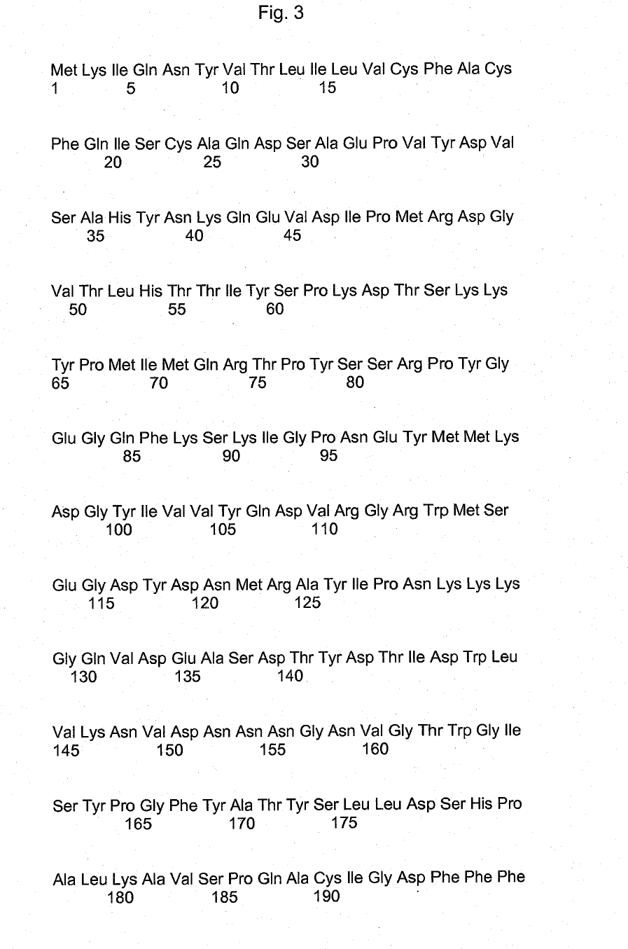

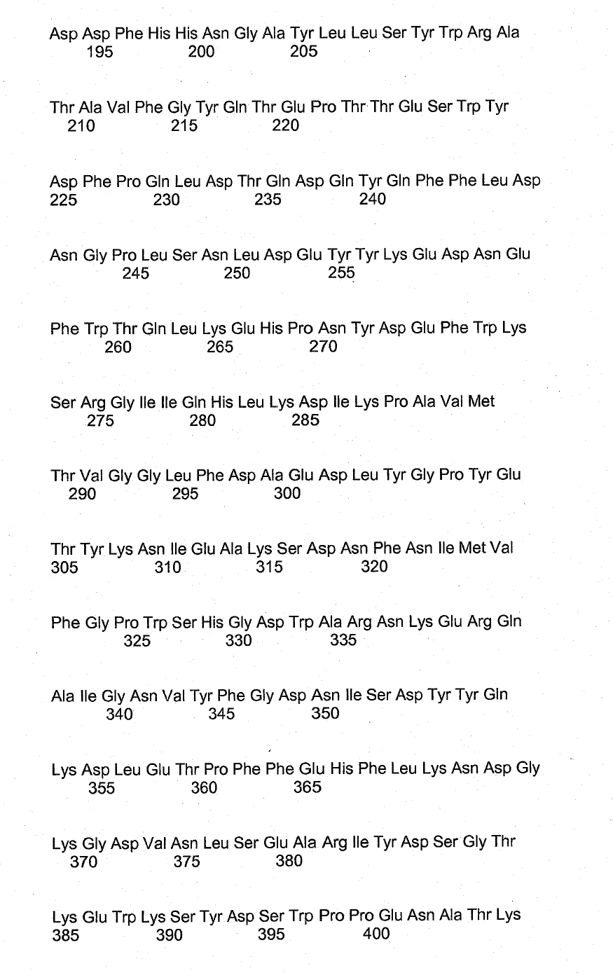

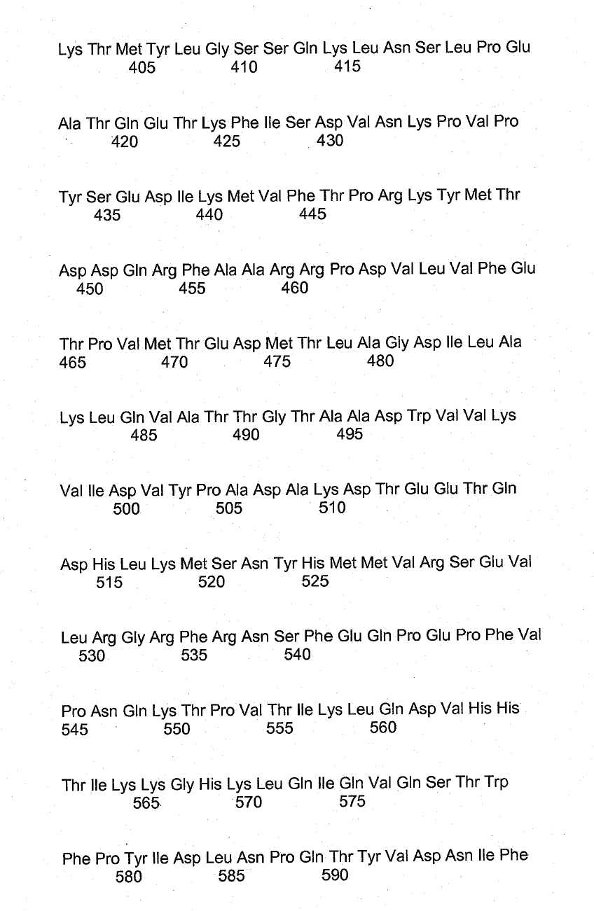

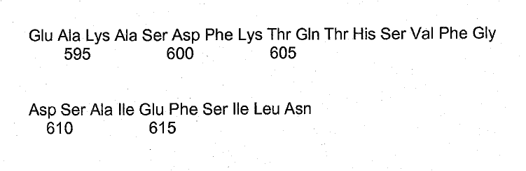

24. A method according to claim 20, wherein cell constituents comprise a protein having a sequence of FIG. 3.


SEQUENCE LISTING (2 sequences)

SEQ ID NO: 1 - protein, 614 aa, Leeuwenhoekiella blandensis
MKNIKNLITAVFLLLVVAAQAQTEAYVREHYDKKEVKIKMRDGVMLHTTIYSPKDKSQEYPILMMRTPYSCAPYGEENFKGRIGPNQFMVEEGNIFVQQDVRGRWNSEGVYDNMRAYIPNKKGKQVDEASDTYDTIDWLVKNVDNNNGNVGTWGISYPGFYATYSLLSEHPALKAVSPQACISDFFFDDFHHNGAFLESYWRAISLFGYMKDEPVTKPWYAFPDLPSQDQYQFFLDAGPLTNLDAYYKEDNVFWQQIKDHPNYDEFWRSRGIIQHLNDIKPAVMVVGGLFDAEDLYGPFNTYRKIEETSDNYNILVYGPWSHGDWARSSERQAIGNVYFGDNLSRDFQQNYETVFFNHFLKNDGEGTIDFPEASMYDSGSREWDSYAQWPPKAAETKSFYLGADQKLTENAIADYANAFVSDPSKPVPYSEDIKMVFTPRKYMTDDQRFAARRPDVLVYETEVLDEDMTITGEILAKLKVATTGTAADWVVKVIDVYPNDAKDYPETQKYLKMGNYHMMVRSEVMRGRFRESFRAPKAFEPNQKTEVDLTLQGVNHTFLKGHKVQIQIQSTWFPLIDRNPQTFVPNIYKASAEDFQKQTHTVFGDSEIQFNVLK

SEQ ID NO: 2 - protein, 618 aa, Crocibacter atlanticus
MKIQNYVTLILVCFACFQISCAQDSAEPVYDVSAHYNKQEVDIPMRDGVTLHTTIYSPKDTSKKYPMIMQRTPYSSRPYGEGQFKSKIGPNEYMMKDGYIVVYQDVRGRWMSEGDYDNMRAYIPNKKKGQVDEASDTYDTIDWLVKNVDNNNGNVGTWGISYPGFYATYSLLDSHPALKAVSPQACIGDFFFDDFHHNGAYLLSYWRATAVFGYQTEPTTESWYDFPQLDTQDQYQFFLDNGPLSNLDEYYKEDNEFWTQLKEHPNYDEFWKSRGIIQHLKDIKPAVMTVGGLFDAEDLYGPYETYKNIEAKSDNFNIMVFGPWSHGDWARNKERQAIGNVYFGDNISDYYQKDLETPFFEHFLKNDGKGDVNLSEARIYDSGTKEWKSYDSWPPENATKKTMYLGSSQKLNSLPEATQETKFISDVNKPVPYSEDIKMVFTPRKYMTDDQRFAARRPDVLVFETPVMTEDMTLAGDILAKLQVATTGTAADWVVKVIDVYPADAKDTEETQDHLKMSNYHMMVRSEVLRGRFRNSFEQPEPFVPNQKTPVTIKLQDVHHTIKKGHKLQIQVQSTWFPYIDLNPQTYVDNIFEAKASDFKTQTHSVFGDSAIEFSILN